(19) United States
(12) Patent Application Publication
(10) Pub. No.: US 2021/0207214 A1
(43) Pub. Date: Jul. 8, 2021

(54) EPIGENETIC METHOD TO ESTIMATE THE EXTRINSIC AGE OF SKIN

(71) Applicant: Conopco, Inc.., d/b/a UNILEVER, Englewood Cliffs, NJ (US)

(72) Inventors: Davd Andrew GUNN, St Neots (GB); Taniya KAWATRA, Twickenham (GB)

(73) Assignee: Conopco, Inc.., d/b/a UNILEVER, Englewood Cliffs, NJ (US)

(21) Appl. No.: 17/057,770

(22) PCT Filed: Jun. 10, 2019
(86) PCT No.: PCT/EP2019/065710

§ 371 Date: Nov. 23, 2020

(30) Foreign Application Priority Data
Jun. 15, 2018 (EP) 18177971.1

(51) Int. Cl. C12Q 1/6876

(52) U.S. Cl. CPC C12Q 1/6876; C12Q 2600/154

(57) ABSTRACT

The invention provides a method for obtaining information useful to determine the extrinsic age of skin of an individual, the method comprising the steps of: (a) obtaining genomic DNA from skin cells derived from the individual; and (b) observing cytosine methylation of >30 CpG loci in the genomic DNA selected from the group consisting of: cg24756227 cg06036239 cg11530289 cg04659582 cg03445800 cg04941246 cg22264616 cg15902864 cg13672200 cg00530720 cg00866690 cg25034941 cg01246665 cg24393844 cg19058262 cg12051116 cg02000606 cg18263166 cg06900899 cg03819134 cg15596932 cg11359720 cg03195377 cg15382568 cg00092551 cg26169991 cg04194664 cg13984289 cg22032385 cg05482603 cg09851620 cg23621013 cg20710730 cg18716076 cg06142351 cg12177909 cg15394860 cg02707854 cg13062888 cg23518497 cg00394718 cg01544580 cg06635832 cg11994639 cg19974120 cg06299192 cg21497480 cg02947450 cg13836638 cg12732514 cg24641302 cg05705140 cg06531870 cg24902858 cg22797031 cg26134692 cg14847243 cg22827250 cg10549088 cg18366919 cg15971980 cg25587920 cg25612391 cg17774851 cg04815577 cg16636721 cg16511229 cg27485152 cg18958844 cg16241033 cg10399789 cg03983058 cg13506653 cg08243094 cg06623668 cg02444978 cg14250984 cg04949225 cg24699871 cg20300541 cg27005906 cg04935109 cg15100426 cg12271419 cg16247183 cg08087655 cg07055302 cg15553500 cg24977027 cg23244910 cg22677715 cg14908170 cg00842231 cg27105183 cg12105671 cg21494776 cg05941864 cg04661001 cg00454305 cg02037307 cg25123102 cg01620208 cg17666539 cg07055879 cg26831119, so that information useful to determine the extrinsic age of the skin of the individual is obtained.

### FIELD OF INVENTION

This invention relates to methods of detecting and analysing patterns of cytosine methylation in genomic DNA. More specifically, it relates to detecting and analysing patterns of cytosine methylation in specific sites in genomic DNA in order to determine the extrinsic age and health of skin.

### BACKGROUND TO INVENTION

It is well known that ageing is a multifactorial process predominantly driven by the age of the individual. Skin ageing in an especially multifactorial phenomenon driven by both intrinsic and extrinsic factors. In terms of intrinsic factors, the chronological age of an individual is the most well-known but other intrinsic factors such as an individual's metabolism, diet, stress and underlying health also contribute to the age if the skin. In addition to these intrinsic factors, the skin is exposed to external challenges such as UV radiation, pollution, drying conditions and extremes of temperature. These extrinsic factors therefore also contribute to the age on an individual's skin.

It is therefore clear that there are two distinct forms of skin age: Extrinsic age, which is dominated by the accumulation of ageing caused by extrinsic factors (i.e. originating from outside the exterior surface of the stratum corneum and that then penetrate into the skin through the stratum corneum), especially sun exposure (photo-ageing); and Intrinsic age, which is the degree of ageing in skin due to factors that originate endogenously; in other words ageing not due to extrinsic factors. For the sake of understanding, it is helpful to consider 2 different types of skin of an individual. One from a site normally protected by clothing (such as the buttock area or upper inner arm area). Another from a sun exposed site (such as the face or back of the hand). The protected site will have far less exposure to extrinsic aging factors and therefore any aging will be due to intrinsic factors. The exposed site will been fully exposed to extrinsic aging factors and therefore the age of this area aging will be due to a combination of both the inherent intrinsic age caused by the intrinsic factors but also the aging due to the extrinsic factors.

The present invention is directed towards the development of an epigenetic method to estimate the extrinsic age of an individual's skin.

DNA methylation is an epigenetic determinant of gene expression. Patterns of CpG methylation are heritable, tissue specific, and correlate with gene expression. The consequence of methylation, particularly if located in a gene promoter, is usually gene silencing. DNA methylation also correlates with other cellular processes including embryonic development, chromatin structure, genomic imprinting, somatic X-chromosome inactivation in females, inhibition of transcription and transposition of foreign DNA and timing of DNA replication. When a gene is highly methylated it is less likely to be expressed. Thus, the identification of sites in the genome containing 5-meC is important in understanding cell-type specific programs of gene expression and how gene expression profiles are altered during both normal development, ageing and diseases such as cancer. Mapping of DNA methylation patterns is important for understanding diverse biological processes such as the regulation of imprinted genes, X chromosome inactivation, and tumor suppressor gene silencing in human cancers.

Horvath S. et al “DNA methylation age of human tissues and cell types” (Genome Biology 14 (2103) R115) reports the use of a transformed version of chronological age that was regressed on CpGs using a penalized regression model (elastic net). The elastic net regression model selected 353 CpGs which were referred to as epigenetic clock CpGs since their weighted average (formed by the regression coefficients) was said to amount to an epigenetic clock. This study is referred to as the “Horvath Study” in this patent.

However, we have now found that for sun-exposed skin sites the predicted ages based on these 353 loci were approximately 9 years younger than their actual (“chronological”) age, indicating they do not detect sun-induced damage in skin. Additionally, sun-protected skin samples were found to have an age 4 years younger than the chronological age which is a underestimation of the age of the sun-protected skin which would be expected to be approximately the same as the chronological age of the subject that the sample was taken from. These 353 loci therefore fail to recognize the difference between photo-damaged and photo-protected skin types, underestimate the age of sun-protected skin, and predict photo-damaged skin as younger than photo-protected. It can therefore be appreciated that this model is not capable of assessing the different forms of aging—extrinsic and intrinsic ageing The present invention therefore aims to address the poor performance of this prior art ageing model and to provide an improved method for evaluating the extrinsic age of skin.

### SUMMARY OF INVENTION

We have surprisingly found that a different, specific set of methylation sites provide enhanced accuracy for the prediction of the extrinsic age of skin.

Accordingly, in a first aspect the invention provides a method for obtaining information useful to determine the extrinsic age of skin of an individual, the method comprising the steps of:

(a) obtaining genomic DNA from skin cells derived from the individual; and

(b) observing cytosine methylation of >30 CpG loci in the genomic DNA selected from the group consisting of CpG locus designation:

cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653 cg08243094 cg06623668 cg02444978 cg14250984
cg04949225 cg24699871 cg20300541 cg27005906 cg04935109 cg15100426 cg12271419
cg16247183 cg08087655 cg07055302 cg15553500 cg24977027 cg23244910 cg22677715
cg14908170 cg00842231 cg27105183 cg12105671 cg21494776 cg05941864 cg04661001
cg00454305 cg02037307 cg25123102 cg01620208 cg17666539 cg07055879 cg26831119,

so that information useful to determine the extrinsic age of the skin of the individual is obtained.

The genomic DNA is obtained from skin cells derived from the individual. The skin sample preferably comprises the epidermis, either alone or in combination with the dermis.

Preferably >40 sites from this group are used, more preferably >45, >50, >55, >60, >65, >70, >75, >80, >85, >90, >95, >100, most preferably all 105 sites of this group are used.

Preferably the loci that are observed are:

cg08243094 cg06623668 cg02444978 cg14250984 cg04949225 cg24699871 cg20300541
cg27005906 cg04935109 cg15100426 cg12271419 cg16247183 cg08087655 cg07055302
cg15553500 cg24977027 cg23244910 cg22677715 cg14908170 cg00842231 cg27105183
cg12105671 cg21494776 cg05941864 cg04661001 cg00454305 cg02037307 cg25123102
cg01620208 cg17666539 cg07055879 cg26831119.

More preferably the loci that are observed are:

cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653.

In an alternative embodiment, the cytosine methylation in the genomic DNA is assessed wherein the genomic DNA is within 20 kBp of any of the CpG locus designations listed above, preferably within 15 kBp, more preferably within 10 kBp, yet more preferably within 5 kBp, even more preferably within 1 kBp, most preferably within 0.5 kBp.

In a second aspect, the invention provides a kit for obtaining information useful to determine the extrinsic age of skin of an individual, the kit comprising:
primers or probes specific for >30 genomic DNA sequences in a biological sample, wherein the genomic DNA sequences comprise CpG loci in the genomic DNA selected from the group consisting only of the following CpG locus designations:

cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653 cg08243094 cg06623668 cg02444978 cg14250984
cg04949225 cg24699871 cg20300541 cg27005906 cg04935109 cg15100426 cg12271419
cg16247183 cg08087655 cg07055302 cg15553500 cg24977027 cg23244910 cg22677715
cg14908170 cg00842231 cg27105183 cg12105671 cg21494776 cg05941864 cg04661001
cg00454305 cg02037307 cg25123102 cg01620208 cg17666539 cg07055879 cg26831119;

and
a reagent used in:
a genomic DNA polymerization process;
a genomic DNA hybridization process;
a genomic DNA direct sequencing process;
a genomic DNA bisulphite conversion process; or
a genomic DNA pyrosequencing process.

Preferably the primers or probes are specific for >40 of the genomic DNA sequences in a biological sample, more preferably >45, >50, >55, >60, >65, >70, >75, >80, >85, >90, >95, >100, most preferably the primers or probes are specific for all 105 sites of this group.

Preferably the primers or probes are specific for genomic DNA sequences in a skin sample, most preferably a skin sample comprising the epidermis, either alone or in combination with the dermis.

Preferably the primers or probes are specific for the following CpG locus designations:

cg08243094 cg06623668 cg02444978 cg14250984 cg04949225 cg24699871 cg20300541
cg27005906 cg04935109 cg15100426 cg12271419 cg16247183 cg08087655 cg07055302
cg15553500 cg24977027 cg23244910 cg22677715 cg14908170 cg00842231 cg27105183
cg12105671 cg21494776 cg05941864 cg04661001 cg00454305 cg02037307 cg25123102
cg01620208 cg17666539 cg07055879 cg26831119.

More preferably the primers or probes are specific for the following CpG locus designations:

cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653.

In an alternative embodiment, the cytosine methylation in the genomic DNA is assessed wherein the genomic DNA is within 20 kBp of the CpG locus designation listed above, preferably within 15 kBp, more preferably within 10 kBp, yet more preferably within 5 kBp, even more preferably within 1 kBp, most preferably within 0.5 kBp.

Preferably the kit comprises a methylation microarray.

Preferably the kit comprises a DNA sequencing method.

### DETAILED DESCRIPTION OF INVENTION AND EXAMPLES

As discussed, the aging process in skin is a highly multifactorial phenomenon that also varies across the body. For example, protected skin is exposed to far fewer insults than exposed skin and it is therefore apparent that different areas of skin from the same individual will have different levels of damage and therefore different “ages”.

In the present invention we consider two forms of skin age: Intrinsic age; and Extrinsic age.

In terms of intrinsic age, the chronological age of an individual is predominant but other endogenous factors such as an individual's metabolism, diet, stress and underlying health also contribute to the age of the skin. Therefore, in the context of the present invention, intrinsic age means the age of the skin caused by endogenous factors.

In terms of extrinsic age, the inherent age will still be a fundamental component but in addition, exogenous factors such as UV radiation, pollution, drying conditions and extremes of temperature will also contribute. Therefore, in the context of the present invention, extrinsic age means the age of the skin caused predominantly by exogenous factors.

For the sake of clarity: Extrinsic age is dominated by the accumulation of ageing caused by extrinsic factors (i.e. originating from outside the exterior surface of the stratum corneum and that then penetrate into the skin through the stratum corneum), especially sun exposure (photo-ageing); whereas Intrinsic age is the degree of ageing in skin due to factors that originate endogenously; in other words ageing not due to extrinsic factors.

The present invention is directed towards the development of an epigenetic method to estimate the extrinsic age of an individual's skin.

Datasets

This application utilised three epigenetic datasets.
Identification: A first dataset was used to identify methylation sites associated with protected and exposed sites in skin.
Training: A second dataset was used to train mathematical models in which the methylation sites identified from the Identification dataset were assessed, those best able to predict the age of the skin were determined, and a predictive model was built.
Testing: Finally, a third test dataset was used to assess the accuracy of these methylation sites in determining the age of the skin samples and whether the use of these methylation sites was more accurate than those identified in the Horvath Study.

The first dataset (Identification) was a single centre, cross-sectional biopsy study involving 24 Chinese and 24 Caucasian female participants in which 24 young and 24 old females had enrolled. Samples of skin were collected from two different areas of each subject: samples from exposed area of the skin; and samples from protected area of the skin. Sites designated as exposed were located on the lower outer arm. Protected sites were located on the upper inner arm, typically half way between the elbow and axilla area.

The second training dataset (Training) was a publicly available dataset (Bormann F. et al: Reduced DNA methylation patterning and transcriptional connectivity define human skin aging. Aging Cell (2016) 1-9. Array express id: EMTAB-4385). The dataset comprised a total of 108 epidermis samples, 48 samples had been isolated from punch biopsies that had been obtained from the outer forearm of 24 young (18-27 years) and 24 old (61-78 years). 60 samples had been obtained as suction blister roofs from the outer forearm of 60 volunteers aged 20-79 years. All volunteers were female, Caucasian, and disease-free.

The final test dataset (Testing) was a publicly available dataset (Vandiver A. R. et al.: Age and sun exposure-related widespread genomic blocks of hypomethylation in nonmalignant skin. Genome Biology (2015) 16:80) Gene Expression Omnibus accession number: GSE51954). The dataset contained epidermal samples (N=38) from 20 Caucasian subjects. Paired punch biopsy samples, 4 mm in diameter, had been collected under local anaesthesia from the outer forearm or lateral epicanthus (exposed area) and upper inner arm (protected area).

Choice of Training and Test Datasets

The choice of datasets was guided by the following criteria. First, the training and test data needed to be from epidermal skin, either skin biopsy or epidermis only. The chosen Training data (Bormann et al.) was from skin biopsy and suction blister of the outer forearm and epidermis samples were available for the Testing (Vandiver et al.) dataset. Second, the Training data needed to be on continuous ages and the Testing data needed to have both exposed and protected samples across both young and old age groups. Third, the mean age in the Training dataset (47 years, standard deviation=21) needed to be, and was, comparable to that of the Testing dataset (51 years, standard deviation=25).

Methylation Data Quality Checks

All three datasets used bisulphite converted DNA hybridized to Infinium 450 k human methylation beadchip.

The methylation data from all DNA samples in the Identification dataset passed quality checks based on three array quality metrics (MAplot, Boxplot, Heatmap). Beta-values were calculated as B=R/R+G and M-values were calculated as M=log 2(R/G), where R represents methylated signals and G unmethylated signals. An offset of 60 was added to the denominator. M-values were used to create the expression matrix. Raw data were normalized using quantile normalization. Beta-values were used for subsequent modelling and filtering the statistical results.

Quality control and pre-processing of the Training dataset was done from raw .idat files in ‘minfi’ R package. Raw data was normalized using Subset-quantile Within Array Normalization (SWAN).

For the Testing dataset, the raw .idat files that are necessary for performing SWAN were unavailable. Therefore, the Illumina pre-processed beta values that were provided were used for subsequent analysis. The quality control and pre-processing applied on the data was also done using ‘minfi’ R package.

Technical Influences on the Data

Exploratory analysis using principle component analysis (PCA) on the Identification dataset was carried out. It was found that the between-array replicates did not cluster together, likely due to batch effect linked to array number. Clustering analysis of the Testing dataset revealed a similar array batch effect. No technical batch effect was seen on the Training dataset.

Batch-Effect Corrected Data

The array batch effects observed in the Identification and Testing datasets was adjusted using the ComBat method (Johnson W. E. et al.: Adjusting batch effects in microarray expression data using empirical Bayes methods. Biostatistics 8(1) (2007) 118-127) following quality control, normalization and averaging of within-array replicates. The resulting datasets after batch correction showed no clustering on array. The remaining biological effects were still present and tended to be the main effects in the data.

CpG Loci Identification

As used herein, CpG loci refer to the unique identifiers found in the Illumina CpG loci database (as described in Technical Note: Epigenetics, CpG Loci Identification ILLUMINA Inc. 2010, https://www.illumina.com/documents/products/technotes/technote_cpg_loci_identification.pdf). These CpG site identifiers therefore provide consistent and deterministic CpG loci database to ensure uniformity in the reporting of methylation data.

Performance of Horvath's epigenetic clock in predicting age of sun-exposed skin The age predictor from the Horvath Study (which uses the 353 CpG sites discussed above) was run against the exposed (Se) and protected (Sp) samples of the Testing dataset. The performance of the Horvath model was assessed using Linear Regression from which an R2 (“pho” or “p”) was obtained. Median Error (Predicted vs. Actual Age) was also calculated. The results are provided in Table 1.

TABLE 1
Predicted ages of exposed and protected skin samples
age using predictor from the Horvath Study.

Predicted age

| Actual age | Exposed (Se) | Protected (Sp) |
|---|---|---|
| 20 | 21.32 | 22.66 |
| 21 | 31.26 | 26.20 |
| 22 | 25.04 | 35.02 |
| 25 | 28.63 | 30.63 |
| 27 | 40.24 | 38.89 |
| 28 | 25.55 | 30.63 |
| 29 | 31.61 | 38.17 |
| 30 | 36.08 | 36.95 |
| 34/30* | 34.77 | 33.73 |
| 65 | 47.49 | 53.96 |
| 65 | 55.71 | 48.37 |
| 67 | 54.71 | 51.31 |
| 69 | 50.26 | 63.49 |
| 70 | 54.79 | 58.56 |
| 72 | 56.99 | 65.79 |
| 74 | 59.80 | 62.51 |
| 83 | 47.39 | 66.47 |
| 84 | 47.76 | 66.82 |
| 90 | 55.96 | 68.15 |
| Average age: 51.32/51.11 | 42.39 | 47.28 |

*Se and Sp samples unpaired. Age of the exposed subject is 34, the age of the protected subject is 30.

It can be seen that for 15 out of the 19 subjects the Horvath model calculated exposed samples as being younger than protected samples which is not correct because samples subjected to exposure such as UV radiation are expected to be older than those protected from UV damage.

Average age acceleration on the predicted age reveals the sun-exposed skin sample to have an age 9 years younger than the chronological age which goes against the known physiology that exposure, especially sun-exposure, causes premature ageing of skin.

Additionally, the protected skin samples were found to have an age 4 years younger than the chronological age which is a underestimation of the age of the protected skin which would be expected to be approximately the same as the chronological age of the person from which the sample was taken.

It can therefore be concluded that the 353 CpG sites from the Horvath Study are not able to recognize the difference between exposed and protected skin types, incorrectly predict sun-damaged skin as younger than sun-protected, and underestimate the age of the protected samples.

It was also found that the 353 CpG sites identified by the Horvath Study performed poorly in terms of the accuracy score for exposed samples.

The accuracy score for exposed samples was:
ρ=0.8 (error=17.6 years).

It can therefore be appreciated that an improved epigenetic method for determining the extrinsic age of skin is required.

Identification of methylation sites associated with exposed sites (from the Identification dataset) A total of 5 comparisons, using different linear models were performed on the normalized batch corrected data for the purpose of generating extrinsic and intrinsic age lists (Table 2). A statistical cut-off set at multiple testing corrected lists (adjust P-value—adjP, benjamini Hochberg)<0.05 together with a delta-beta>=0.05 was applied.

A high number of differentially methylated CpG sites were detected for the comparison of young versus old in exposed sites (Comparison 1: n=10,649). Relatively fewer differentially methylated CpG sites were identified for the comparison of age group versus site interaction (Comparison 5: n=233).

TABLE 2
Statistical results. Number of differentially methylated sites for
each of the 5 comparisons with adjusted p-value cut-off of 0.05.

| | | Number of differentially |
|---|---|---|
| | Comparison | methylated CpG sites detected |
| 1 | Young vs. Old exposed sites | 10,649 |
| 2 | Young vs. Old protected sites | 3,545 |
| 3 | Protected vs. exposed (Young) | 3,714 |
| 4 | Protected vs. exposed (Old) | 7,053 |
| 5 | Age group: Site interaction | 233 |

Extrinsic Site List

To identify CpG sites that capture extrinsic aging, Comparison 1 (Young vs. Old exposed sites) results were filtered to remove probes not changing by site in young or old in the same direction (Comparisons 3 & 4), to remove any intrinsic aging changes not associated with extrinsic ageing factors, especially exposure.

The resulting list was 2,259 CpG sites. PCA analysis on these 2,259 sites allowed identification of sites contributing to maximum variance in classifying exposed sites into young and old groups across both ethnicities. After testing several thresholds, PCA loading cut-off of 0.024 was applied to the first component resulting in 310 probes. These 310 methylation probes best captured ageing changes occurring in exposed skin and hence reflective of extrinsic ageing. The 233 probes from Comparison 5 were also included, as they demonstrated a greater change with age in the exposed samples than the protected samples indicating they also reflected extrinsic ageing. The final extrinsic age list comprises 505 CpG sites.

Extrinsic Age Predictor from Exposed Sites

The 505 CpG sites identified to capture extrinsic age changes from the Identification dataset were used to build an extrinsic age model in which the same elastic net as that used in the Horvath Study was utilised on the Training dataset with 10 sets of size n/10 (train on 9 datasets and test on 1). These were repeated 10 times and a mean “accuracy” for each iteration was obtained to give a model for calculating age, and a coefficient for each probe.

Lists of predictors were arrived at by running several iterations of the model. The first iteration identified the best set of predictors. For each subsequent iteration, the identified predictors from the previous iteration were excluded from the training set to identify the next-best set of predictors. The iterations were repeated until the predictive accuracy, measured in terms of rho and error margin was found to be less accurate than that of the Horvath model as described above.

For the extrinsic sites, 3 iterations were performed. The first identified 73 sites, the second identified 32 sites, the third identified 26 as shown in Table 3.

Resultant models where the sites from each of these 3 iterations were removed from the final extrinsic age list of 505 CpG sites were used to estimate the age of the exposed samples from the Testing dataset. The results are shown in Table 4. In addition, the average ages for both sun-protected and sun exposed samples were calculated for the resultant models. The results are shown in Table 5. The accuracy of the model using 353 sites from Horvath study for predicting extrinsic age is also shown in Tables 4 and 5 (in italics) for reference.

TABLE 3
Predictor sets for Extrinsic age scores.

| Iteration 1 (73 sites) | Iteration 2 (32 sites) | Iteration 3 (26 sites) |
|---|---|---|
| cg24756227 | cg08243094 | cg25076881 |
| cg06036239 | cg06623668 | cg19263548 |
| cg11530289 | cg02444978 | cg09098707 |
| cg04659582 | cg14250984 | cg19160624 |
| cg03445800 | cg04949225 | cg21145416 |
| cg04941246 | cg24699871 | cg08805037 |
| cg22264616 | cg20300541 | cg06621027 |
| cg15902864 | cg27005906 | cg26798452 |
| cg13672200 | cg04935109 | cg24438334 |
| cg00530720 | cg15100426 | cg13936863 |
| cg00866690 | cg12271419 | cg08145067 |
| cg25034941 | cg16247183 | cg12883980 |
| cg01246665 | cg08087655 | cg10031651 |
| cg24393844 | cg07055302 | cg16609957 |
| cg19058262 | cg15553500 | cg19519747 |
| cg12051116 | cg24977027 | cg26837962 |
| cg02000606 | cg23244910 | cg10086659 |
| cg18263166 | cg22677715 | cg11160654 |
| cg06900899 | cg14908170 | cg21498785 |
| cg03819134 | cg00842231 | cg09937500 |
| cg15596932 | cg27105183 | cg10931190 |
| cg11359720 | cg12105671 | cg01025233 |
| cg03195377 | cg21494776 | cg15768226 |
| cg15382568 | cg05941864 | cg27546066 |
| cg00092551 | cg04661001 | cg13001963 |
| cg26169991 | cg00454305 | cg15108410 |
| cg04194664 | cg02037307 | |
| cg13984289 | cg25123102 | |
| cg22032385 | cg01620208 | |
| cg05482603 | cg17666539 | |
| cg09851620 | cg07055879 | |
| cg23621013 | cg26831119 | |
| cg20710730 | | |
| cg18716076 | | |
| cg06142351 | | |
| cg12177909 | | |
| cg15394860 | | |
| cg02707854 | | |
| cg13062888 | | |
| cg23518497 | | |
| cg00394718 | | |
| cg01544580 | | |
| cg06635832 | | |
| cg11994639 | | |
| cg19974120 | | |
| cg06299192 | | |
| cg21497480 | | |
| cg02947450 | | |
| cg13836638 | | |
| cg12732514 | | |
| cg24641302 | | |
| cg05705140 | | |
| cg06531870 | | |
| cg24902858 | | |
| cg22797031 | | |
| cg26134692 | | |
| cg14847243 | | |
| cg22827250 | | |
| cg10549088 | | |
| cg18366919 | | |
| cg15971980 | | |
| cg25587920 | | |
| cg25612391 | | |
| cg17774851 | | |
| cg04815577 | | |
| cg16636721 | | |
| cg16511229 | | |
| cg27485152 | | |
| cg18958844 | | |
| cg16241033 | | |
| cg10399789 | | |
| cg03983058 | | |
| cg13506653 | | |

TABLE 4
Accuracy of models

| | R2 values |
|---|---|
| | (sun exposed |
| Model | sites) |
| Model using 505 sites (final extrinsic age list) | 0.86 |
| Model using 423 sites (73 sites from iteration 1 removed) | 0.85 |
| Model using 400 sites (32 sites from iteration 2 removed) | 0.78 |
| Model using 374 sites (26 sites from iteration 3 removed) | 0.77 |
| Model using 353 sites from Horvath study | 0.80 |

According to the accuracy measures shown in Table 4 the models of extrinsic age that included the sites identified in iteration 1 (R2=0.86) and iteration 2 (R2=0.85) performed with higher accuracy than the models using the 353 Horvath sites (which was R2=0.80). However, once the sites identified in iterations 1 and 2 had been removed, the remaining 400 sites (which included those from iteration 3) performed with lower accuracy than the 353 Horvath sites. Therefore, the 105 sites of iterations 1 and 2 were better at predicting extrinsic age than the Horvath model.

It is expected that the extrinsic age of samples from sun-exposed sites will be higher than for samples from sun-protected sites. As can be seen from Table 5, this is the case for all of the models from this study. However, this is not the case for the Horvath model, which shows the opposite outcome (i.e. samples from sun-exposed sites have a lower average age than those from sun-protected sites). This demonstrates that the models described herein are better than the Horvath model in predicting extrinsic age.

TABLE 5
Average age for models

| | Average age | | |
|---|---|---|---|
| | Sun- | Sun- | Differ- |
| Model | exposed | protected | ence |
| Model using 505 sites | 55.58 | 45.12 | 10.46 |
| (final extrinsic age list) | | | |
| Model using 423 sites | 51.57 | 40.60 | 10.96 |
| (73 sites from iteration 1 removed) | | | |
| Model using 400 sites | 50.11 | 37.97 | 12.14 |
| (32 sites from iteration 2 removed) | | | |
| Model using 374 sites | 54.48 | 40.79 | 13.69 |
| (26 sites from iteration 3 removed) | | | |
| Model using 353 sites from Horvath study | 42.39 | 47.28 | −4.89 |

It can therefore be seen that the use of CpG sites selected from those of iterations 1 and 2 as shown in Table 3 delivers better accuracy when determining the extrinsic age of skin. Therefore, the present invention provides >30 of these 105 sites for use in predicting the extrinsic age of skin. The invention also provides the 32 sites of iteration 2 as a preferred group. The invention further provides the 73 sites of iteration 1 as the most preferred group.

It is an alternative of the invention that the foregoing CpG sites may also be replaced and the closest gene used instead.

Table 6 provides annotations of the 105 sites identified in Iterations 1 & 2 (as described in Price et al. Epigenetics & Chromatin 2013, 6:4, “Additional annotation enhances potential for biologically-relevant analysis of the Illumina Infinium HumanMethylation450 BeadChip array” using Human Genome version HG19), including the closest gene names.

TABLE 6
Annotations of 105 CpG sites identified in Iterations 1 & 2

| CpG | Chromosome | Position of | Closest |
|---|---|---|---|
| Site ID | No. | Methylation on Chr | Gene Name |
| cg24756227 | chr5 | 1406177 | BC034612 |
| cg06036239 | chr1 | 59234929 | JUN |
| cg11530289 | chr8 | 67350852 | ADHFE1 |
| cg04659582 | chr2 | 231276073 | SP100 |
| cg03445800 | chr3 | 523012 | AK126307 |
| cg04941246 | chr3 | 55518131 | WNT5A |
| cg22264616 | chr11 | 32410090 | WT1 |
| cg15902864 | chr5 | 33504903 | TARS |
| cg13672200 | chr12 | 54387530 | MIR196A2 |
| cg00530720 | chr11 | 46368045 | DGKZ |
| cg00866690 | chr1 | 2066631 | PRKCZ |
| cg25034941 | chr13 | 47326127 | AK123654 |
| cg01246665 | chr15 | 93258479 | FAM174B |
| cg24393844 | chr12 | 68115110 | BC035381 |
| cg19058262 | chr19 | 1231292 | C19orf26 |
| cg12051116 | chr2 | 122238129 | CLASP1 |
| cg02000606 | chr7 | 87103624 | ABCB4 |
| cg18263166 | chr7 | 92533866 | CDK6 |
| cg06900899 | chr2 | 241393833 | MIR149 |
| cg03819134 | chr5 | 957584 | L0C100506688 |
| cg15596932 | chr17 | 41836563 | SOST |
| cg11359720 | chr21 | 45246441 | LOC284837 |
| cg03195377 | chr8 | 142289782 | SLC45A4 |
| cg15382568 | chr22 | 25800078 | LRP5L |
| cg00092551 | chr7 | 127371565 | SND1 |
| cg26169991 | chr7 | 155049620 | AX746871 |
| cg04194664 | chr17 | 43716617 | C17orf69 |
| cg13984289 | chr17 | 10220829 | MYH13 |
| cg22032385 | chr2 | 236721769 | AK000798 |
| cg05482603 | chr10 | 118607718 | ENO4 |
| cg09851620 | chr1 | 95403214 | LOC729970 |
| cg23621013 | chr7 | 135433353 | FAM180A |
| cg20710730 | chr17 | 46705577 | HOXB9 |
| cg18716076 | chr5 | 50677808 | ISL1 |
| cg06142351 | chr2 | 8683945 | ID2 |
| cg12177909 | chr11 | 14691596 | PDE3B |
| cg15394860 | chr11 | 2017084 | AK311497 |
| cg02707854 | chr19 | 17600122 | SLC27A1 |
| cg13062888 | chr1 | 18325742 | IGSF21 |
| cg23518497 | chr12 | 60298928 | SLC16A7 |
| cg00394718 | chr8 | 120684921 | ENPP2 |
| cg01544580 | chr17 | 46180269 | CBX1 |
| cg06635832 | chr5 | 154654216 | KIF4B |
| cg11994639 | chr7 | 1997028 | MAD1L1 |
| cg19974120 | chr21 | 42839625 | pp9284 |
| cg06299192 | chr3 | 154513392 | MME |
| cg21497480 | chr8 | 29783819 | LOC286135 |
| cg02947450 | chr10 | 22766861 | LOC100499489 |
| cg13836638 | chr22 | 42679804 | LOC388906 |
| cg12732514 | chr17 | 77726237 | ENPP7 |
| cg24641302 | chr1 | 165087025 | LMX1A |
| cg05705140 | chr2 | 242945114 | BC101234 |
| cg06531870 | chr13 | 89037260 | SLITRK5 |
| cg24902858 | chr11 | 120973231 | TECTA |
| cg22797031 | chr1 | 170630070 | PRRX1 |
| cg26134692 | chr2 | 101778863 | BC077729 |
| cg14847243 | chr3 | 184104362 | CHRD |
| cg22827250 | chr5 | 134363823 | AK026965 |
| cg10549088 | chr3 | 64277154 | PRICKLE2 |
| cg18366919 | chr19 | 15344364 | EPHX3 |
| cg15971980 | chr6 | 150254442 | BC040898 |
| cg25587920 | chr2 | 85604366 | ELMOD3 |
| cg25612391 | chr19 | 19216451 | SLC25A42 |
| cg17774851 | chr5 | 92929319 | NR2F1 |
| cg04815577 | chr5 | 51898 | PLEKHG4B |
| cg16636721 | chr21 | 47920571 | DIP2A |
| cg16511229 | chr7 | 130126153 | MEST |
| cg27485152 | chr8 | 142311034 | LOC731779 |
| cg18958844 | chr2 | 55509779 | PRORSD1P |
| cg16241033 | chr10 | 14050455 | FRMD4A |
| cg10399789 | chr1 | 92945668 | GFI1 |
| cg03983058 | chr16 | 77369724 | ADAMTS18 |
| cg13506653 | chr4 | 54965863 | GSX2 |
| cg08243094 | chr1 | 26930419 | MIR1976 |
| cg06623668 | chr19 | 13138816 | NFIX |
| cg02444978 | chr7 | 16438128 | ISPD |
| cg14250984 | chr11 | 62342677 | EEF1G |
| cg04949225 | chr13 | 50796845 | BCMS |
| cg24699871 | chr6 | 30123191 | TRIM10 |
| cg20300541 | chr17 | 15295802 | Metazoa_SRP |
| cg27005906 | chr12 | 6540162 | CD27 |
| cg04935109 | chr3 | 187086530 | RTP4 |
| cg15100426 | chr2 | 219187432 | PNKD |
| cg12271419 | chr8 | 22855616 | RHOBTB2 |
| cg16247183 | chr1 | 225865110 | AK124056 |
| cg08087655 | chr11 | 122073541 | MIR100HG |
| cg07055302 | chr2 | 55507730 | PRORSD1P |
| cg15553500 | chr4 | 41880987 | BC025350 |
| cg24977027 | chr2 | 88469347 | THNSL2 |
| cg23244910 | chr6 | 106434169 | PRDM1 |
| cg22677715 | chr2 | 162284644 | TBR1 |
| cg14908170 | chr2 | 240405317 | HDAC4 |
| cg00842231 | chr19 | 11352474 | C19orf80 |
| cg27105183 | chr17 | 71898861 | LINC00469 |
| cg12105671 | chr19 | 7852207 | CLEC4GP1 |
| cg21494776 | chr19 | 10397780 | ICAM4 |
| cg05941864 | chr1 | 22893978 | EPHA8 |
| cg04661001 | chr19 | 19217217 | SLC25A42 |
| cg00454305 | chr16 | 1429905 | UNKL |
| cg02037307 | chr5 | 134363562 | AK026965 |
| cg25123102 | chr20 | 44879723 | CDH22 |
| cg01620208 | chr8 | 142311010 | LOC731779 |
| cg17666539 | chr19 | 7927207 | EVI5L |
| cg07055879 | chr7 | 143747474 | OR2A5 |
| cg26831119 | chr4 | 111550830 | PITX2 |

1. A method for obtaining information useful to determine the extrinsic age of skin of an individual, the method comprising the steps of:
(a) obtaining genomic DNA from skin cells derived from the individual; and
(b) observing cytosine methylation of >30 CpG loci in the genomic DNA selected from the group consisting of:
cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653 cg08243094 cg06623668 cg02444978 cg14250984
cg04949225 cg24699871 cg20300541 cg27005906 cg04935109 cg15100426 cg12271419
cg16247183 cg08087655 cg07055302 cg15553500 cg24977027 cg23244910 cg22677715
cg14908170 cg00842231 cg27105183 cg12105671 cg21494776 cg05941864 cg04661001
cg00454305 cg02037307 cg25123102 cg01620208 cg17666539 cg07055879 cg26831119,
so that useful to determine e age of the skin of the individual is obtained.

2. A method according to claim 1 wherein >40 sites from the group are used, more preferably >45, >50, >55, >60, >65, >70, >75, >80 >85, >90, >95, >100, most preferably all 105 sites.

3. A method according to claim 1 wherein the loci that are observed are the following CpG loci:
cg08243094 cg06623668 cg02444978 cg14250984 cg04949225 cg24699871 cg20300541
cg27005906 cg04935109 cg15100426 cg12271419 cg16247183 cg08087655 cg07055302
cg15553500 cg24977027 cg23244910 cg22677715 cg14908170 cg00842231 cg27105183
cg12105671 cg21494776 cg05941864 cg04661001 cg00454305 cg02037307 cg25123102
cg01620208 cg17666539 cg07055879 cg26831119.

4. A method according to claim 1 wherein the loci that are observed are the following CpG loci:
cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653.

5. A kit for obtaining information useful to determine the extrinsic age of skin of, an individual, the kit comprising:
primers or probes specific for >30 genomic DNA sequences in a biological sample, wherein the genomic DNA sequences comprise CpG loci in the genomic DNA selected from the group consisting only of the following CpG locus designations:
cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653 cg08243094 cg06623668 cg02444978 cg14250984
cg04949225 cg24699871 cg20300541 cg27005906 cg04935109 cg15100426 cg12271419
cg16247183 cg08087655 cg07055302 cg15553500 cg24977027 cg23244910 cg22677715
cg14908170 cg00842231 cg27105183 cg12105671 cg21494776 cg05941864 cg04661001
cg00454305 cg02037307 cg25123102 cg01620208 cg17666539 cg07055879 cg26831119;
and
a reagent used in:
a genomic DNA polymerization process;
a genomic DNA hybridization process;
a genomic DNA direct sequencing process;
a genomic DNA bisulphite conversion process; or
a genomic DNA pyrosequencing process.

6. A kit according to claim 5 wherein the primers or probes are specific for >40 of the genomic DNA sequences in a biological sample, more preferably >45, >50, >55, >60, >65, >70, >75, >80, >85, >90, >95, >100, most preferably all 105.

7. A kit according to claim 5 wherein the primers or probes are specific for genomic DNA sequences in a skin sample.

8. A kit according to claim 5 wherein the primers or probes are specific for the following CpG locus designations:
cg08243094 cg06623668 cg02444978 cg14250984 cg04949225 cg24699871 cg20300541
cg27005906 cg04935109 cg15100426 cg12271419 cg16247183 cg08087655 cg07055302
cg15553500 cg24977027 cg23244910 cg22677715 cg14908170 cg00842231 cg27105183
cg12105671 cg21494776 cg05941864 cg04661001 cg00454305 cg02037307 cg25123102
cg01620208 cg17666539 cg07055879 cg26831119.

9. A kit according to claim 5 wherein the primers or probes are specific for the following CpG locus designations:
cg24756227 cg06036239 cg11530289 cg04659582 cg03445800 cg04941246 cg22264616
cg15902864 cg13672200 cg00530720 cg00866690 cg25034941 cg01246665 cg24393844
cg19058262 cg12051116 cg02000606 cg18263166 cg06900899 cg03819134 cg15596932
cg11359720 cg03195377 cg15382568 cg00092551 cg26169991 cg04194664 cg13984289
cg22032385 cg05482603 cg09851620 cg23621013 cg20710730 cg18716076 cg06142351
cg12177909 cg15394860 cg02707854 cg13062888 cg23518497 cg00394718 cg01544580
cg06635832 cg11994639 cg19974120 cg06299192 cg21497480 cg02947450 cg13836638
cg12732514 cg24641302 cg05705140 cg06531870 cg24902858 cg22797031 cg26134692
cg14847243 cg22827250 cg10549088 cg18366919 cg15971980 cg25587920 cg25612391
cg17774851 cg04815577 cg16636721 cg16511229 cg27485152 cg18958844 cg16241033
cg10399789 cg03983058 cg13506653.
